(19) United States
(12) Patent Application Publication
(10) Pub. No.: US 2003/0148966 A1
(43) Pub. Date: Aug. 7, 2003

(54) Method and composition for inducing apoptosis in cells

(76) Inventors: Hiremagalur Jayaram, Indianapolis, IN (US); Georg Krupitza, Vienna (AT); Thomas Szekeres, Vienna (AT); Michael Grusch, Vienna (AT)

Correspondence Address: KOHN & ASSOCIATES, PLLC, Suite 410, 30500 Northwestern Highway, Farmington Hills, MI, 48334, US

(21) Appl. No.: 10/347,990

(22) Filed: Jan. 20, 2003

Related U.S. Application Data
(60) Provisional application No. 60/353,938, filed on Jan. 30, 2002.

(51) Int. Cl. A61K 31/7076; A61K 31/7052

(52) U.S. Cl. 514/42; 514/48

(57) ABSTRACT

There is provided a composition including an ATP-inducing compound or ATP and an effective amount of cell death-inducing drug. Also provided is the method of inducing apoptosis in apoptosis-inducible cells by administering to the apoptosis inducible cells a composition including an effective amount of a cell death-inducing compound and an ATP-inducing compound or ATP. The present invention also provides a method of suppressing necrosis in cells by administering an effective amount of an ATP-inducing compound or ATP to a cell population, thereby inhibiting necrosis.

### CROSS-RELATED REFERENCE SECTION

This application claims the benefit of priority under 35 U.S.C. Section 119(e) of U.S. Provisional Patent Application No. 60/353,938, filed Jan. 30, 2002, which is incorporated herein by reference.

### BACKGROUND OF THE INVENTION

1. Field of the Invention

Generally, the present invention relates to methods and compositions for inducing apoptosis in cells for use as therapeutics or in conjunction therewith.

2. Description of Related Art

Apoptosis, or programmed cell death, is a naturally occurring process of cell suicide that plays a crucial role in the development and maintenance of metazoans by eliminating superfluous or unwanted cells. In cell culture experiments, cell death can be initiated by activation of specific receptors like TNF-receptor or fas/Apo receptor or alternatively simply by withdrawal of serum or appropriate growth factors (Enari et al., 1995; Evan et al., 1992; Jimenez et al., 1995; Ju et al., 1995; Tamm and Kikuchi, 1991, 1993; Tamm et al., 1991, 1992; Tewari and Dixit, 1995). There is a series of criteria used to distinguish programmed death (apoptosis) from necrosis. At the morphological level, apoptosis is characterized by nuclear changes, i.e. aggregation of chromatin at the nuclear membrane, membrane blebbing without loss of integrity, chromatin fragmentation, and formation of membrane bound vesicles (apoptotic bodies; Steller, 1995). These processes are not associated with a disintegration of organelles like mitochondria. Apoptotic bodies are finally phagocytosed by adjacent cells or macrophages.

Necrosis might be initiated by chemical or physical insults including osmotic imbalance, or energy deprivation. It has been proposed that due to loss of ATP a breakdown of the cytoskeleton occurs that leads to bleb like structures which are prone to shear stress (Kabakov and Gabai, 1994). Further morphological changes include swelling of cells and disintegration of organelles. It is evident from the results presented above that not all criteria of either apoptosis or necrosis are found during the death of AKR-2B fibroblasts after serum removal. The osmolarities of the serum-containing and serum-free media do not significantly differ, and moreover, no cell swelling was observed.

Cell death constitutes one of the key events in biology. At least two modes of cell death can be distinguished: apoptosis and necrosis. Apoptosis is a strictly regulated (programmed) device responsible for the ordered removal of superfluous, aged, misbehaving, or damaged cells. Every second, several millions of cells of the human body undergo apoptosis; i.e., in conditions of homeostasis, each mitosis is compensated by one event of apoptosis. It is presumed that all cells of the human body possess the intrinsic capacity of undergoing apoptosis, even in the absence of de novo protein synthesis, which suggests that all structures and processes required for at least one pathway to apoptosis are ubiquitously present (and probably necessary for cell survival).

Macromolecular synthesis may be required for certain agents to cause apoptosis, either because they have different pathways or because of linkages to the pre-existing proteins particular to these agents. Disturbances in apoptosis regulation illustrate the importance of apoptosis for normal homeostasis. An abnormal resistance to apoptosis induction correlates with malformations, autoimmune diseases, or cancer due to the persistence of superfluous, cell-specific immunocytes, or mutated cells, respectively. In contrast, enhanced apoptotic decay of cells participates in acute pathologies (infection by toxin-producing microorganisms, ischemia-reperfusion damage, or infarction) as well as in chronic diseases (neurodegenerative and neuromuscular diseases, AIDS). Although apoptosis is necessary for both health and disease, necrosis is always the outcome of severe and acute injury: i.e. abrupt anoxia, sudden shortage of nutrients such as glucose, or extreme physicochemical injury (heat, detergents, strong bases etc). In contrast to necrosis, apoptosis involves the regulated action of catabolic enzymes (proteases and nucleases) within the limits of a near-to-intact plasma membrane. Apoptosis is commonly accompanied by a characteristic change of nuclear morphology (chromatin condensation, pyknosis, karyorrhexis) and of chromatin biochemistry (step-wise DNA fragmentation). It also involves the activation of specific cysteine proteases (caspases) that cleave after aspartic acid residues. Caspases catalyze a highly selective pattern of protein degradation. Subtle changes in the plasma membrane occur before it ruptures. Thus, the surface exposure of phosphatidylserine residues (normally on the inner membrane leaflet) allows for the recognition and elimination of apoptotic cells by their healthy neighbors, before the membrane breaks up and cytosol or organelles spill into the intercellular space and elicit inflammatory reactions (6). Moreover, cells undergoing apoptosis tend to shrink while reducing the intracellular potassium level.

In contrast to apoptosis, necrosis does not involve any regular DNA and protein degradation pattern and is accompanied by swelling of the entire cytoplasm (oncosis) and of the mitochondrial matrix, which occur shortly before the cell membrane ruptures.

Compounds shown to be effective in the treatment of cancer cells typically affect such cells by inducing maturation (i.e., slowing growth) of the cells or by killing the cells (i.e., necrosis), because the compound itself is toxic. Compounds which slow cancer cell growth or are toxic to the cancer cells are often disadvantageous because the compounds themselves often adversely affect normal cells.

It has been discovered that cancer cells can be induced to kill themselves (i.e., to undergo programmed cell death, hereinafter referred to as “apoptosis”). Compounds, which can induce cancer cells to kill themselves, are less likely to adversely affect the patient because the compound affects normal cells to significantly less than cancer cells (i.e., normal cells are able to recover at doses which are effective for the treatment of cancer cells).

More specifically, the process of necrosis is characterized by the inflammation of a colony of cells that include both cancer and normal cells. When cells are contacted with a necrosis-inducing agent, the cells break down into relatively large fragments with DNA typically withstanding any significant fragmentation (i.e. DNA being typically greater than 100,000 bases). Thus, necrosis is a collective experience in a cell population such that both cancer cells and normal cells are affected.

The mechanism of apoptosis is not clearly understood. It is believed that apoptosis arises due to a change in the gene expression in the cell causing the cell to program and induce its own death. The result is a breakup of the genetic messenger, DNA, into smaller enveloped components that can be absorbed by adjacent cells without harmful effect.

More specifically, apoptosis is characterized by the selective programmed destruction of cancer cells into relatively small fragments with DNA becoming highly fragmented. During apoptosis, cell shrinkage and internucleasomal DNA cleavage occurs, followed by the fragmentation of the DNA. Eventually the cell disintegrates into small fragments.

There is a significant difference in the results achieved by necrosis as compared with apoptosis. The cellular material remaining after necrosis is large and relatively difficult for unaffected cells to assimilate. In the aftermath of apoptosis, because the remaining material is in relatively small units, they are readily canabolized by unaffected cells. Therefore, apoptosis-inducing agents possess significant advantages over compounds that induce necrosis. Such agents are not only selective for cancer cell destruction, but also enable the fragmented cellular material to be safely assimilated by the body.

Benzamide riboside has been shown to be a compound capable of inducing differentiation of cancer cells. More specifically, benzamide riboside has been shown to be cytotoxic to S49.1 lymphoma cells by Karsten Krohn et al., J. Med. Chem., Vol. 35, 11-517 (1992) and to human myelogenous leukemia cells by Hiremagalur N. Jayaram et al., Biochem. Biophys. Res. Commun., Vol. 186, No. 3, pp. 1600-1606 (1992), each of which is incorporated herein by reference.

As indicated in H. N. Jayaram et al., benzamide riboside inhibits the enzyme inosine 5′-monophosphate dehydrogenase (IMP dehydrogenase), which is necessary for cell growth. However, in vitro inhibition of IMP dehydrogenase requires very high concentrations of benzamide riboside, suggesting that the compound may require conversion to a different form to exert IMP dehydrogenase inhibitory activity. Accordingly, benzamide riboside has been described as a pro-drug.

More recently, Kamran Gharehbaghi, et al., Int. J. Cancer, Vol. 56, pp. 892-899 (1994) disclosed that benzamide riboside exhibited significant cytotoxicity against a variety of human tumor cells in culture through a derivative of benzamide riboside, benzamide adenine dinucleotide (BAD).

The references discussed above show that benzamide riboside acts through its dinucleotide derivative to induce cell death. There has not been shown a mechanism by which to control whether cell death occurs via necrosis or apoptosis. It would therefore be useful to develop a composition, which in conjunction with benzamide riboside or another cell death-inducing compound, to induce a specific type of cell death. More specifically, it would be beneficial to develop a compound that induces apoptosis.

### SUMMARY OF THE INVENTION

According to the present invention, there is provided a composition including a compound or ATP, and an effective amount of cell death-inducing drug. Also provided is the method of inducing apoptosis in apoptosis-inducible cells by administering to the apoptosis inducible cells, a composition including an effective amount of a cell death-inducing compound and an ATP-inducing compound or ATP. The present invention also provides a method of suppressing necrosis in cells by administering an effective amount of an ATP-inducing compound, or ATP to a cell population, thereby inhibiting necrosis.

### BRIEF DESCRIPTION OF THE FIGURES

Other advantages of the present invention will be readily appreciated as the same becomes better understood by reference to the following detailed description when considered in connection with the accompanying drawings wherein:

FIGS. 1A and B are photographs of Western blots showing the cleavage of the caspase substrate (poly(ADP-ribose) polymerase (PARP) (FIG. 1A) and gelsolin (FIG. 1B) following treatment of HL-60 cells with increasing doses of benzamide riboside;

FIGS. 2A-C are electron microscopic images of apoptotic and necrotic HL-60 cells treated with saline (FIG. 2A), 5 μM benzamide riboside (FIG. 2B) and 20 μM benzamide riboside (FIG. 2C);

FIGS. 3A-D are photographs showing induction of cell death modes by benzamide riboside;

FIGS. 4A-C are graphs showing a correlation of nucleoside levels with cell death modes;

FIG. 5 is a bar graph showing modulations of cell death by adenosine and glucose;

FIGS. 6A and B are photographs of comet assays at neutral pH showing the induction of DNA damage in HL-60 cells by benzamide riboside; and

FIG. 7 is a graph showing cell death modulation by 3-aminobenzamide.

### DETAILED DESCRIPTION OF THE INVENTION

The present invention provides a composition and method for inducing apoptosis in cells, which are apoptosis inducible. This is accomplished by administering an effective amount of a cell death-inducing drug and either ATP or an ATP-inducing compound.

The phrase “cell death-inducing drug” is intended to mean, but is not limited to, a compound that induces or otherwise causes cell death when administered. Cell death can occur either via necrosis or apoptosis. Examples of such compounds include, but are not limited to benzamide riboside and tiazofurin.

Benzamide riboside (BR) is a C-nucleoside¹, that has recently been characterized as an inosine 5′-monophosphate dehydrogenase (IMPDH)-inhibitor^{2,3}. This enzyme catalyzes the conversion of IMP to xanthosine 5″-monophosphate (XMP) and is the rate limiting enzyme in de novo guanylate biosynthesis⁴. The activity of this enzyme is significantly increased in tumor cells and therefore, considered to be a potential target for cancer chemotherapy⁵. Tiazofurin (TR), which is homologous to BR, was found to inhibit the growth of the human myelogenous leukemia K562⁶ and human promyelocytic leukemia HL-60 cells⁷. TR is an inhibitor of IMPDH⁸ and Phase I/II clinical trials conducted with this compound in acute myelogenous leukemia patients, indicated a significant reduction in leukemic cell burden⁹⁻¹¹. BR, exhibited stronger anti-proliferative activity in the K562 cells than its structural homologue TR¹² and was shown to induce apoptosis in HL-60¹³ and N.1 ovarian carcinoma cells^{13,14}. Higher BR-concentrations, however, provoked necrosis, which is a common phenomenon of pro-apoptotic drugs¹⁵⁻¹⁷ and limits chemotherapy because of non-specific drug toxicity. Over-dosing results in necrosis and spilling of intracellular fluids into the peri-cellular space, leading to inflammatory responses with wide ranging destruction of surrounding tissues. Therefore, it is of prime interest to develop strategies to suppress necrosis and favor apoptosis. Interestingly, both cell death modes, apoptosis and necrosis, were discussed to partly share similar early pathways¹⁷⁻¹⁹.

It was postulated that BR exerts its anti-tumor effects due to IMPDH inhibition^{2,12}. Therefore, dGTP and other dNTP levels were analyzed and correlated with cell death modes. The necrotic trigger of high BR-concentrations was identified as DNA-clastogenic activity, which subsequently led to ATP depletion. ATP levels are the key factor deciding the death modes, because apoptosis is energy-(ATP-) dependent, whereas, necrosis is not. Therefore, in order to reduce non-specific toxicity by drug-overload, ATP levels are kept high to specifically promote apoptosis and prevent necrosis.

Benzamide riboside has been described as an inhibitor of cell growth and/or differentiation by inhibiting IMP dehydrogenase, which catalyzes the formation of xanthine 5′-monophosphate (XMP) from inosine 5′-monophosphate (IMP). The inhibition of IMP dehydrogenase adversely affects the synthesis of guanine nucleotides and thus, limits the cells' ability to grow and/or differentiate.

In accordance with the present invention, benzamide riboside, when delivered in a specified concentration range, can affect the DNA of apoptosis-inducible cancer cells to cause a change in the genetic makeup, which programs the cell to undergo apoptosis. In particular, the administration of benzamide riboside appears to result in a sustained expression of c-myc proto-oncogene and a down regulation of the cell cycle gene cdc25A, which is believed to interrupt the cell cycle progression causing conditions suitable for apoptosis.

Benzamide riboside has also been described as an inhibitor of cell growth and/or differentiation by inhibiting IMP dehydrogenase, which catalyzes the formation of xanthine 5′-monophosphate (XMP) from inosine 5′-monophosphate (IMP). The inhibition of IMP dehydrogenase adversely affects the synthesis of guanine nucleotides and thus, limits the cells' ability to grow and/or differentiate.

In accordance with the present invention, benzamide riboside, when delivered in a specified concentration range, can affect the DNA of apoptosis-inducible cancer cells to cause a change in the genetic makeup. This change programs the cell to undergo apoptosis. In particular, the administration of benzamide riboside appears to result in a sustained expression of c-myc proto-oncogene, and a down regulation of the cell cycle progression gene cdc25A.

More specifically, the administration of benzamide riboside to select cancer cells (i.e. cancer cells which can be induced to undergo apoptosis) is characterized by DNA fragmentation as evidenced by a laddering effect on polyacrylamide gel and a concurrent down-regulation of the G1 phase specific gene, cdc25A, expression in cancer cells.

The cancer cells, which can be treated in accordance with the present invention, are those that are capable of being induced to undergo chronic apoptosis (i.e. capable of being programmed themselves). Some cancer cells (i.e. human myelogenous leukemia K562 cells) possess the gene bcr-abl, which prevents apoptosis even in the presence of an apoptosis-inducing agent. Unless the anti-apoptosis gene can be regulated, such cancer cells (i.e. human myelogenous leukemia K562 cells) cannot be induced to undergo apoptosis by the administration of benzamide riboside. It has also been observed that cells in which the gene bcl-2 expression levels are increased and/or the gene p53 is expressed, is also resistant to the induction of apoptosis.

There are, however, many types of cancer cells that are susceptible to apoptosis through the administration of benzamide riboside and are therefore, within the scope of the present invention. Such cells include ovarian carcinoma, breast carcinoma, CNS carcinoma, renal carcinoma, lung cancer cells, leukemia cells such as human promyelocytic leukemia cells, and the like.

The amount of benzamide riboside administered to the apoptosis-inducible cancer cells is at least 5 μmoles/l, based on a cancer cell population of approximately one million cells (hereinafter referred to as “per one million cancer cells”). A preferred concentration range for benzamide riboside is from about 5 μmoles/l to 25 micromoles per one million cancer cells. Most preferred is a concentration range of from about 10 micromoles to 20 micromoles, per one million cancer cells.

Benzamide riboside can be administered to a warm-blooded animal in the form of pharmaceutically acceptable salts. Included among these salts are sodium sulfate, ammonium sulfate, ammonium chloride, calcium chloride, calcium sulfate, and the like.

Benzamide riboside can be administered in combination with pharmaceutically acceptable carriers in the form of a pharmaceutically acceptable composition. Such carriers include mannose, glucose, and balanced salt solutions. The compositions containing benzamide riboside including carriers, can be lyophilized by adding sterile water as described in Lawrence A. Trissel et al., “The Handbook on Injectable Drugs”, 8th Edition, published by the American Society of Hospital Pharmacists (1994), incorporated herein by reference.

The compositions are preferably administered intravenously or orally. Oral administration of the composition is preferably carried out, by using conventional inert carriers such as mannitol, sodium chloride, and/or the calcium carbonate salt form of benzamide riboside.

Benzamide riboside and salts thereof are administered in a therapeutically effective dose, depending on the cancer to be treated. Generally, the dosage of benzamide riboside and salts thereof is in the range of from about 1 to 10 mg/kg/day, which is administered in at least one dosage form per day. The daily dosage is preferably administered orally, subcutaneously, or parenterally, including intravenous, intraarterial, intramuscular, intraperitoneally, and intranasal administration, as well as intrathecal and infusion technique for five to ten days. When administered intravenously, the preferred daily dosage period is from about one to two hours.

In a preferred form of the invention, benzamide riboside is encapsulated in liposomes prepared according the Francis Zoke, Jr. et al., Eroc. Natl. Acad. Sci. Vol. 75, 4194-4198 (1978), incorporated herein by reference.

A typical product of benzamide riboside encapsulated in liposomes, contains 33 μmol of cholesterol in 1.0 ml of aqueous phase (phosphate buffered saline), and 3 ml of solvent (e.g. diethyl ether, isopropyl ether, halothane, or trifluorotrichloroethane). These ratios are maintained for maximum capture. When vesicles are formed from Pal₂ PpdCho, an additional 3 ml of chloroform or 0.8 ml of methanol is added to the preparation, and the vesicles are allowed to remain at 45° C. for at least 30 minutes after evaporation of the solvent. To determine the amount of encapsulated benzamide riboside or salt thereof, the vesicles are dialyzed overnight against 300 volumes of phosphate buffered saline.

The phrase “cell death-inducible cell” is intended to mean a cell, which is capable of being induced to die. In other words, the cell must be able to be influenced to begin the cell death pathway.

The phrase “ATP-inducing drug” is intended to be defined as a compound which induces, or causes to be produced, ATP. An ATP-inducing drug can cause a cell to increase ATP production within the cell, either directly or indirectly. For example, the drug, can either directly increase ATP production, by causing an increase in intracellular ATP, or the drug can increase the production of ATP precursors. The ATP-inducing drug can be any drug known to those of skill in the art, or can be a gene therapy, which enables the cell to increase ATP production. The drug preferably increases ATP production to at least normal levels. It is not necessary for the drug to increase ATP production above normal levels; however, such production is not detrimental. The drug must also be effective at the site of cells being treated. The drug must elevate the ATP levels of the target tissue and it must be timed such that the effective level is reached in conjunction with, the effective dose of the cell death inducer. Hence, the pharmacokinetics for the pair of drugs administered must co-react accordingly. Such pharmacological coordination is known to those skilled in the art.

Gene therapy as used herein, refers to the transfer of genetic material (e.g DNA or RNA) of interest into a host to treat or prevent a genetic or acquired disease or condition phenotype. The genetic material of interest encodes a product (e.g. a protein, polypeptide, peptide, functional RNA, anti-sense), whose production in vivo is desired. For example, the genetic material of interest can encode a hormone, receptor, enzyme, polypeptide, or peptide of therapeutic value. Alternatively, the genetic material of interest encodes a suicide gene. For a review see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy, cells are removed from a patient and while being cultured, are treated in vitro. Generally, a functional replacement gene is introduced into the cell via an appropriate gene delivery vehicle/method (transfection, transduction, homologous recombination, etc.), and an expression system as needed, and then the modified cells are expanded in culture and returned to the host/patient. These genetically reimplanted cells have been shown to express the transfected genetic material in situ.

In in vivo gene therapy, target cells are not removed from the subject, rather the genetic material to be transferred, is introduced into the cells of the recipient organism in situ that is within the recipient. In an alternative embodiment, if the host gene is defective, the gene is repaired in situ [Culver, 1998]. These genetically altered cells have been shown to express the transfected genetic material in situ.

The gene expression vehicle is capable of delivery/transfer of heterologous nucleic acid into a host cell. The expression vehicle may include elements to control targeting, expression, and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein, the expression vehicle may, as needed, not include the 5′UTR and/or 3′UTR of the actual gene to be transferred, and only include the specific amino acid coding region.

The compound of the present invention is administered and dosed in accordance with good medical practice, taking into account the clinical condition of the individual patient, the site and method of administration, scheduling of administration, patient age, sex, body, weight, and other factors known to medical practitioners. The pharmaceutically “effective amount” for purposes herein is thus determined by such considerations as are known in the art. The amount must be effective to achieve improvement including, but not limited to, improved survival rate or more rapid recovery, or improvement or elimination of symptoms, and other indicators as are selected as appropriate measures by those skilled in the art.

In the method of the present invention, the compound of the present invention can be administered in various ways. It should be noted that it can be administered as the compound, or as pharmaceutically acceptable salt, and can be administered alone or as an active ingredient, in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and in particular, mammals including man. The pharmaceutically acceptable carriers, diluents, adjuvants, and vehicles, as well as implant carriers, generally refer to inert, non-toxic solid or liquid fillers, diluents, or encapsulating material not reacting with the active ingredients of the invention.

It is noted that humans are treated generally longer than the mice or other experimental animals exemplified herein, which treatment has a length proportional to the length of the disease process and drug effectiveness. The doses may be single doses or multiple doses over a period of several days, but single doses are preferred.

The doses may be single doses or multiple doses over a period of several days. The treatment generally has a length proportional to the length of the disease process, drug effectiveness, and the patient species being treated.

When administering the compound of the present invention parenterally, it will generally be formulated in a unit dosage injectable form (solution, suspension, emulsion). The pharmaceutical formulations suitable for injection include, sterile aqueous solutions or dispersions, and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil, and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate, and gelatin. According to the present invention, however, any vehicle, diluent, or additive used, would have to be compatible with the compounds.

Sterile injectable solutions can be prepared by incorporating the compounds. utilized in practicing the present invention, in the required amount of the appropriate solvent with variations of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants, or targeted delivery systems, such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques, which deliver the compound orally or intravenously and retain the biological activity, are preferred.

In one embodiment, the compound of the present invention can be administered initially by intravenous injection to bring blood levels to a suitable level. The patient's levels are then maintained by an oral dosage form, although other forms of administration, dependent upon the patient's condition, and as indicated above, can be used. The quantity to be administered will vary for the patient being treated and will vary from about 100 ng/kg of body weight to 100 mg/kg of body weight per day and preferably will be from 1.0 mg/kg to 10 mg/kg per day.

The composition of the present invention includes a cell death-inducing drug and an ATP-inducing drug. In the preferred embodiment, the cell death-inducing drug and an ATP-inducing drug are included in a single composition. Alternatively, the two drugs can be administered separately. If the two drugs are administered separately, then the ATP-inducing drug is administered first and followed by the cell death-inducing drug.

The composition of the present invention can be used to cause a cell to undergo apoptosis. The method of causing such apoptosis includes administering the composition to the cells to be treated.

The above discussion provides a factual basis for the use of the compound and method for inducing apoptosis in cells. The methods used with a utility of the present invention, can be shown by the following non-limiting examples and accompanying figures.

### EXAMPLES

### Example 1

Chemicals

BR was synthesized as described earlier¹. Polyclonal Caspase 3 antibody was purchased from Santa Cruz, adenosine, glucose, 3-amino benzamide (3-AB), all trans retinoic acid (ATRA) and cyanide (KCN) were from Sigma (St. Louis, Mo., USA).

Cell Culture

The HL-60 human acute promyelocytic leukemia cell line was from ATCC (Rockville, Md., USA). Cells were grown in RPMI 1640 medium (Gibco, Grand Island, N.Y., USA) with 10% heat inactivated FCS (Boehringer Mannheim GmbH, Mannheim, Germany) and 2 nM L-Glutamine (Gibco, Gaithersburg, Md., USA) in humidified atmosphere with 5% CO₂ at 37° C.

Hoechst 33258 Propidium Iodide (HOPI) Double Staining

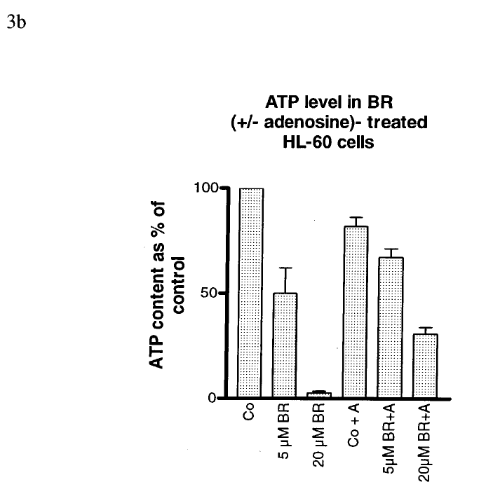

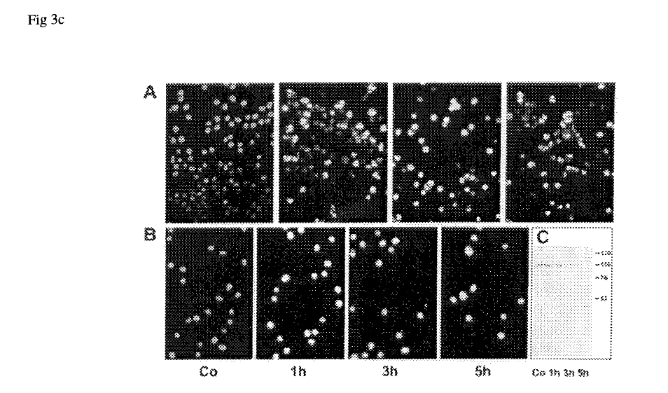

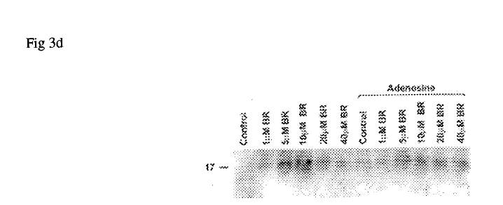

Hoechst 33258 (HO; Sigma) and propidium Iodide (PI; Sigma) were added directly to the culture medium to final concentrations of 5 μg/ml and 2 μg/ml, respectively. After an incubation period of 1 hour at 37° C., the cells were examined under a Zeiss Axiovert 35 fluorescence microscope with DAPI filters. Cells were photographed on Kodak Ektachrome P1600 film (Eastman Kodak Company, Rochester, N.Y., USA) and viable, apoptotic, and necrotic cells were counted manually. The Hoechst dye stains the nuclei of all cells, and therefore, allows monitoring nuclear changes associated with apoptosis, such as chromatin, condensation, and nuclear fragmentation (FIG. 3c). PI, on the other hand, is excluded from viable and early apoptotic cells, and consequently PI uptake indicates loss of membrane integrity characteristic of necrotic and late apoptotic cells. In combination with fluorescence microscopy, the selective uptake of the two dyes allows to monitor the induction of apoptosis in intact cultures and to distinguish it from non-apoptotic cell death (necrosis). Necrosis is characterized in this system by nuclear PI uptake without chromatin condensation or nuclear fragmentation.

Electron Microscopy

For transmission electron microscopy (TEM) cells treated with PBS (controls), 5, or 20 μM BR for 24 hours were fixed with 2.5 percent glutaraldehyde (in 0.1 M sodium cacodylate buffer with 4% sucrose, pH 7.2) for 45 minutes, washed in sodium cacodylate buffer and post-fixed in 2% osmium tetroxide (in 0.1 M sodium cacodylate buffer with 4% sucrose) for 45 minutes. Following several washes, the cells were concentrated by centrifugation at 150 g for 5 minutes, dehydrated in a graded series of ethanol, washed in propylene oxide, embedded in Epon (Serva, Germany), and sectioned at about 70 nm. The ultra-thin sections were stained with uranyl acetate/lead citrate for observation with a Zeiss EM 902 transmission electron microscope.

ATP Assay

ATP content was measured with the ATP bioluminescence assay kit HS II from Boehringer Mannheim (Roche Molecular Biochemicals, Mannheim, Germany). Cells were treated with adenosine and BR for 16 hours, and subsequently their viability was measured by trypan blue exclusion. There were <10% dead cells in each sample. 2.5 million cells were pelleted for each measurement and resuspended in 50 μl dilution buffer. An equal amount of cell lysis reagent was then added, and after an incubation period of 5 minutes at room temperature, the samples were transferred to microtiter plates. A Luciferase reagent (100 μl) was added and the signal was detected immediately on a lumi Imager F1 (Roche). Experiments were done in triplicates, and the values of treated samples calculated as percent of untreated controls.

Western Blots

Cells from treated and untreated cultures were sedimented, washed in cold PBS, and lysed in SDS sample buffer (25 mM TRIS pH 6.8, 3% SDS, 10% glycerol, 36 mM DTT, 0.925 mM EDTA). Equal amounts of protein (calculated with the Dot Metric Protein Assay Kit from Novus Molecular, San Diego, Calif., USA) were loaded onto 10% or 15% polyacrylamide gels. Proteins were electrophoresed at 80 V for 2 to 3 hours, and then transblotted onto PVDF membranes (Hybond P, Amersham International, UK) at 80 V for 2 hours²⁰. Membranes were quenched in PBS with 0.5% skim milk and 0.05% Tween 20 for 1 hour, incubated with primary antibodies (mouse monoclonal anti PARP C-2-10 used 1:2000, mouse monoclonal anti-gelsolin used 1:2000, Sigma; rabbit polyclonal anti caspase 3 antibody used 1:1000, St. Cruz) overnight at 4° C., and with horseradish peroxidase, conjugated secondary antibody for 2 hours at room temperature. The ECL kit (Amersham International, UK) was used for blot development; chemiluminescence was detected on Kodak Xomat UV films.

Deoxyribonucleotide Extraction and Measurement

DNTPs were extracted as described previously²¹ with trichloracetic acid (TCA) 10% final concentration), followed by neutralization by triocylamine and 1,1,2-trichlorotrifluoroethane (1:4) mixture. The TCA extract was dried using a Speedvac drying system at room temperature and, if necessary, stored at −20° C. until analysis. The assay for dNTP, which is based on the original DNA-polymerase assay²², was optimized by the use of 96-well plates²³ and tailor-made oligonucleotides^{24,25}, and was performed as previously described for dCTP²¹. After reconstitution of the samples in Hepes-buffered assay buffer (pH 7.3) at 10⁷ cells/ml, samples and standards (0, 1, 2.5 and 5 pmol dNTP) were added to diethylaminoethyl (DEAE) filter plates (Millipore, Ettenleur, The Netherlands). This was followed by the addition of demi water (up to 30 μl if a reaction mix, consisting of 10 μl [8-³H]dATP (25 μM; 1.6 Ci/mmol; 0.04 μCi/μl) for detection of dCTP, dTTP and dGTP and 10 μl[CH₃-³H]dTTP (25 μM; 1.6 Ci/mmol; 0.04 μCi/μl) for dATP detection, 5 μl appropriate oligonucleotide (6 μM. consisting of a primer attached to one of four possible templates specially designed for the detection of one of the four dNTPs), 5 μl Klenow DNA pol I and 50 μl assay buffer. The filterplates were gently vortexed and incubated at room temperature for 2 hours. The wells were washed, the wet filters punched out and radioactivity counted as described²¹.

Comet Assay and Statistical Analysis

Neutral and alkaline single cell gel electrophoresis (SCGE) assays were carried out following the protocol described by Singh, et al.²⁶. To measure DNA double strand breaks, electrophoresis was carried out under neutral conditions at pH 7.5²⁷. To analyze single strand breaks, electrophoresis was carried out under alkaline conditions at pH 13.0²⁸. HL-60 cells were treated with BR and adenosine for 16 hours, then the viability for the cells was determined with trypan blue. All cultures which were used for Comet analysis, had a viability of >90%. Pellets obtained upon centrifugation were mixed with 100 μl low melting agarose (0.5%, 37° C.), and spread on agarose coated slides according to Klaude et al.²⁹. Subsequently, the slides were exposed to lysis buffer²⁶ and transferred to neutral and alkaline electrophoresis buffer, respectively, for 40 minutes to allow unwinding of DNA. Thereafter, electrophoresis was carried out for 40 minutes (300 mA, 25 V) at pH 7.5 and 13.0, respectively. Finally the slides were stained with ethidium bromide and evaluated under a fluorescence microscope (Nikon Model: 027012), with an automated image analysis system³⁰. For each experimental point, three cultures were evaluated and from each culture, the tail lengths of 50 cells were determined. Statistically significant (p<0.05) differences were determined with one-way ANOVA³⁰.

Results

The BR Concentrations Determines the Type of Cell Death

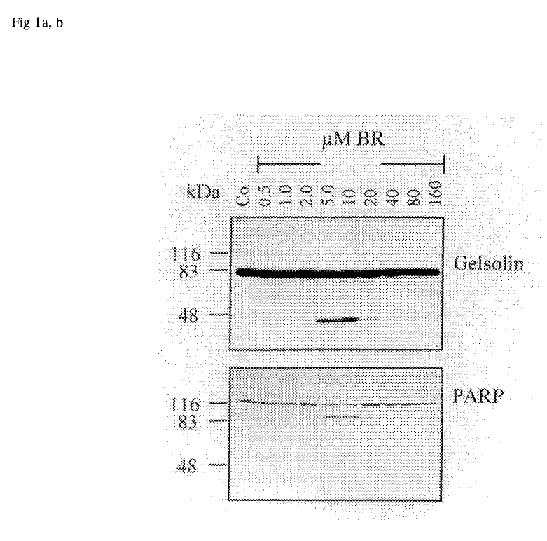

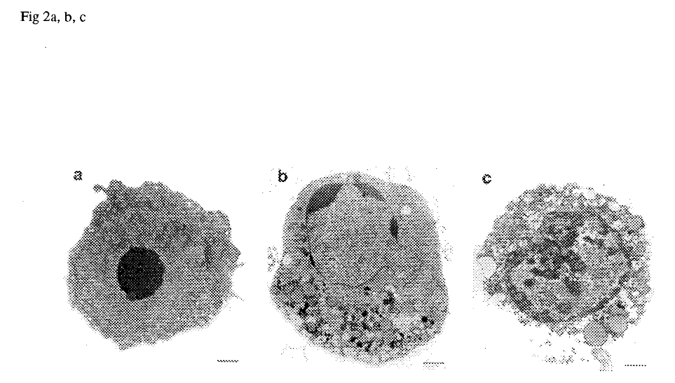

Treatment of HL-60 cells with increasing doses of BR induced cell death, which was analyzed by trypan blue staining (data shown)³¹. To further discriminate the type of cell death, the integrity of poly(ADP-ribose) polymerase [PARP] and gelsolin was examined by Western blotting (FIG. 1). PARP and Gelsolin became signature-specifically fragmented by Caspase 3 upon induction of apoptosis. Addition of 0.5 μM, 1 μM, or 2 μM BR, neither induced cell death, nor gelosin cleavage, whereas concentrations of 5 μM or 10 μM BR caused apoptosis, which was evidenced by degradation of gelsolin into a 41 kD fragment³², and of PARP into an 89 kD fragment (FIG. 1a, b). Further increase of the BR-concentrations to 20 μM led to an increase of the fraction of necrotic cells and reduced the fraction of apoptotic cells, which was also reflected by the lack of PARP and gelsolin fragmentation.

More specifically, FIG. 1 shows the cleavage of the capase substrate poly(ADP-ribose) polymerase (PARP) (FIG. 1A), and gelsolin (FIG. 1B) following treatment of HL-60 cells with increasing doses of BR. Controls were treated with saline for 24 hours and western blots were performed as described herein. The 89 kD cleavage product of PARP and the 41 kD cleavage product of gelsolin could be detected upon treatment with 5 μM and 10 μM BR, and diminished in response to higher doses of BR.

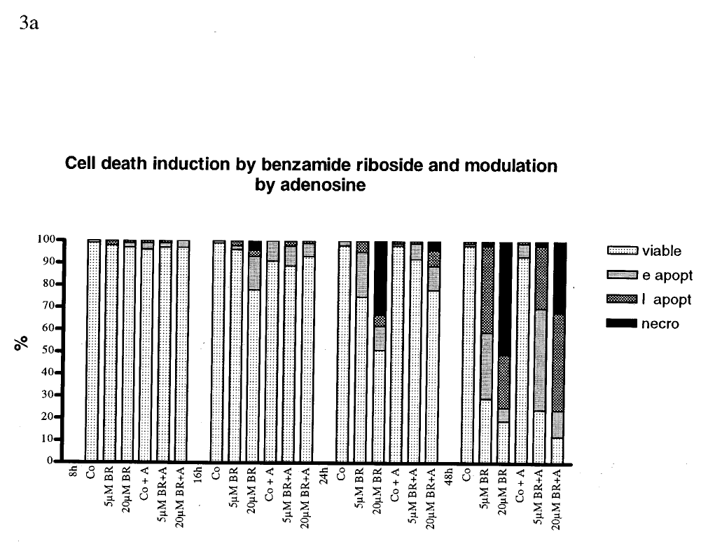

HL-60 cells treated with 5 μM or 20 μM BR for 48 hours, exhibited typical apoptotic or necrotic morphologies, respectively, which was examined by electron microscopy (FIG. 2). The untreated control cell (FIG. 2a) was characterized by an intact cell membrane and nuclear envelope and a normal chromatin distribution. HL-60 cells treated with 5 μM BR (FIG. 2b) still maintained intact membranes, but electron dense chromatin marginated at the nuclear envelope as a hallmark of apoptosis. The vacuoles in the cytoplasm seemed to enlarge during BR treatment, indicating the activation of a detoxifying defense mechanism. HL-60 cells treated with 20 μM BR (FIG. 2c) exhibited typical necrotic morphology, such as disrupted membranes and cloudy chromatin.

FIG. 2 shows HL-60 cells were treated with saline (FIG. 2A), 5 μM BR (FIG. 2B), and 20 μM BR (FIG. 2C) for 24 hours, and prepared for electron microscopial analysis as described in “Methods”. FIG. 2A shows an intact cell morphology; FIG. 2B shows an apoptotic cell with typical DNA condensation and margination at the nuclear envelope; and FIG. 2C shows a necrotic cell exhibiting cloudy chromatin and destructed organelles and membranes. The bars at the lower right corners indicated 1.1 μm.

Measurement of dNTP- and ATP-Levels

The dNTP levels were determined after 16-hours of treatment. At this time point, cells were still alive and membranes intact, preventing non-specific loss of dNTP's³³. Table 1 shows that 5 μM or 20 μM BR, repressed dGTP levels to similar extent (approximately 53% of control). The cellular dGTP concentrations did not correlate with induction of apoptosis or necrosis by 5 μM or 20 μM BR, respectively, after 16 hours, 24 hours, or 48 hours of treatment (compare with FIG. 3a). Whereas, dCTP (89% of control)- and dATP (81% of control)-levels were in the range of control when cells were treated with 5 μM BR, exposure to 20 μM BR, caused a drop in dCTP and dATP levels to 37% and 33%, respectively.

It was assumed that ATP level is a determinant of cell death modes^{15,34-36}. Hence, we determined the ATP levels in HL-60 cells (FIG. 3b) after treatment with 5 μM (which causes apoptosis), and after exposure to 20 μM BR, (which mostly causes necrosis) (FIG. 3a). If ATP determines the type of cell death, then it has to be a regulatory parameter before cell death (apoptosis and/or necrosis) occurs. Therefore, the intercellular ATP pools were examined after 16 hours of treatment (FIG. 3b), when the cell membranes were still intact, and no non-specific loss of nucleotides took place. Treatment with a membrane permeable ATP-precursor, adenosine, was expected to replenish the intracellular pools of ATP and to inhibit necrosis. In fact, the addition of 800 μM adenosine rescued the ATP levels in 20 μM BR treated cells to 31% of the control value, whereas a dramatic ATP drop 2.7% of control was seen, when cells were exposed to 20 μM only (FIG. 3b). In the adenosine-treated cells, necrosis was indeed inhibited (FIG. 3a). FIG. 3c shows HOPI double stained viable cells, early and late apoptosis cells, and necrotic HL-60 cells, which were exposed to 5 μM and 20 μM BR, with or without adenosine (panel A). For reasons of comparison, panel B depicts necrotic HL-60 cells, which underwent heat shock treatment (55° C.) for increasing times, and panel C demonstrates the lack of PARP cleavage after heat shock (FIG. 3c). Re-directing necrosis to apoptosis was also reflected by Caspase 3 cleavage to its active form in the presence of 800 μM adenosine in HL-60 cells, which were treated with 20 μM BR (FIG. 3d). Whereas, Caspase 3 activation culminated (p20 kD fragment) after exposure to 5 μM and 10 μM BR after 24 hours, activation was inhibited after exposure to 20 μM or 40 μM BR. In contrast, Caspase 3 became more activated by 20 μM and 40 μM BR in presence of 800 μM adenosine. Lower adenosine levels had no effect, probably due to limits in cellular take up or due to specific degradation by adenosine deaminase, which is saturated at 800 μM.

FIG. 3A shows cells were treated with saline (Co), 800 μM adenosine (Co+A), 5 μM BE (+/−adenosine), 20 μM BR (+/−adenosine) for 8, 16, 24, and 48 hours. Cells were harvested and stained with HO-PI, applied on glass slides, allowed to settle to the surface, and then counted under a microscope using a DAPI filter, and cell death was determined, wherein “e apopt” early apoptotic cells; “1 apopt’ late apoptotic cells; and “necro” necrotic cells. Statistical analysis by t-test confirmed that the differences between apoptosis-and necrosis-rates after treatment with BR (+/−adenosine) for 48 hours were significant (p<0.05).

FIG. 3B shows cells were treated with saline (Co), adenosine (A), 5 μM and 20 μM BR (+/−adenosine) for 16 hours, which was a time point at which cellular membranes were still intact to avoid leaking. Cells were harvested and the intercellular ATP content was measured as described in herein. Statistical analysis by t-test confirmed that the differences between ATP-levels after treatment with BR (+/−adenosine) were significant (p<0.05).

FIG. 3C shows micrographs of HL-60 cells stained with Hoechst 33258 (HO) and propidium iodide (PI) after treatment with saline, 5 μM BR 20 μM BR, and 20 μM BR+800 μM adenosine (FIG. 3A) for 48 hours (1^{st}, 2^{nd}, 3^{rd}, and 4^{th} slides, from left to right, respectively). FIG. 3B shows HL-60 cells, which were exposed to 55° C. heat shock for increasing times. The nuclei of viable cells stain blue (the cytoplasm remains invisible). In early phase of apoptosis, condensed chromatin is visible as small round bodies, which usually stain more intense blue with HO. Late apoptotic cells exhibit similar chromatin condensation, but the color shifts pink due to PI intrusion through leaky membranes as a consequence of apoptosis progression. Upon 20 μM BR treatment, or in response to heat shock, increasing numbers of cells show a pink color but lack apoptotic (condensed) chromatin (necrotic cells). FIG. 3C shows HL-60 cells, which were exposed to heat shock treatment (55° C.) for one, three, and five hours, and PARP expression and degradation was monitored by western blotting. There is no apoptosis specific cleavage of PARP into the 89 kD product detectable.

FIG. 3D shows the processing of Caspase 3 into the activated p20 polypeptide HL-60 cells, which were treated with saline (control) or increasing doses of BR+/−800 μM adenosine. 5 μM and 10 μM BR, which mainly induce apoptosis, triggering processing of caspase 3.20 μM and 40 μM BR mainly provokes necrosis. This is also reflected by the reduced levels of activated caspase 3. In the presence of adenosine, 20 μM and 40 μM BR activate caspase 3 and also induce apoptosis. Equal sample loading was controlled by Ponceau S staining.

ATP-, dATP-, and dCTP-Levels Correlate with Apoptosis

To elucidate, whether a direct correlation exists between nucleotide pools and death modes, ATP, dATP, and dCTP levels were plotted in combination with the corresponding apoptosis- and necrosis-rates. For improved comprehension, the ATP-, dATP-, dCTP- levels and death data were summarized in Table 2 and graphically compared the inter-relationships of total cell deaths, the apoptotic- and the necrotic subtypes with nucleotide levels from differently treated cells. It can be seen in FIGS. 4a, 4b, and 4c that ATP-, dATP-, dCTP-levels directly correlate with apoptosis rates and inversely with rates, when cell death was induced by BR (not however, in non-induced cells, such as control or adenosine control; not shown in FIGS. 4a-4c). There was no correlation of ATP or dATP levels with total cell deaths (apoptosis+necrosis).

Apoptosis is an energy dependent process, because to maintain membrane integrity ATP is required. Since adenosine restored the ATP pool and prevented necrosis, glucose was anticipated to prevent necrosis^{18,36-38} and to determine death modes. In fact, 100 mM glucose nearly completely inhibited necrosis of HL-60 cells induced by treatment with 20 μM BR, and instead favored apoptosis, which was determined by HOPI double staining^{33,33,39}. In these experiments spontaneous cell death of controls exhibited an apoptosis:necrosis—ratio A:N=3.6:1.20 μM BR resulted in a ratio A:N=1:3.4, which was converted by the addition of glucose to a ratio A:N=7.8:1.

FIG. 4 shows the result of HL-60 cells, which were treated with 5 μM BR (5), 20 μM BR (20), +/−adenosine (+A), for 16 hours, which was a time point when membranes were still intact, and at which cell death modes are already determined, to measure ATP-(a), dATP-levels (c), and for 48 hours to analyze cell death modes. These graphs demonstrate that ATP, dATP, and dCTP levels correlate directly with the percentages of apoptotic cells, whereas, ATP, dATP, and dCTP levels correlate indirectly with the percentages of necrotic cells.

Adenosine Prevents ATRA- and KCN-Triggered Necrosis in Favor of Apoptosis

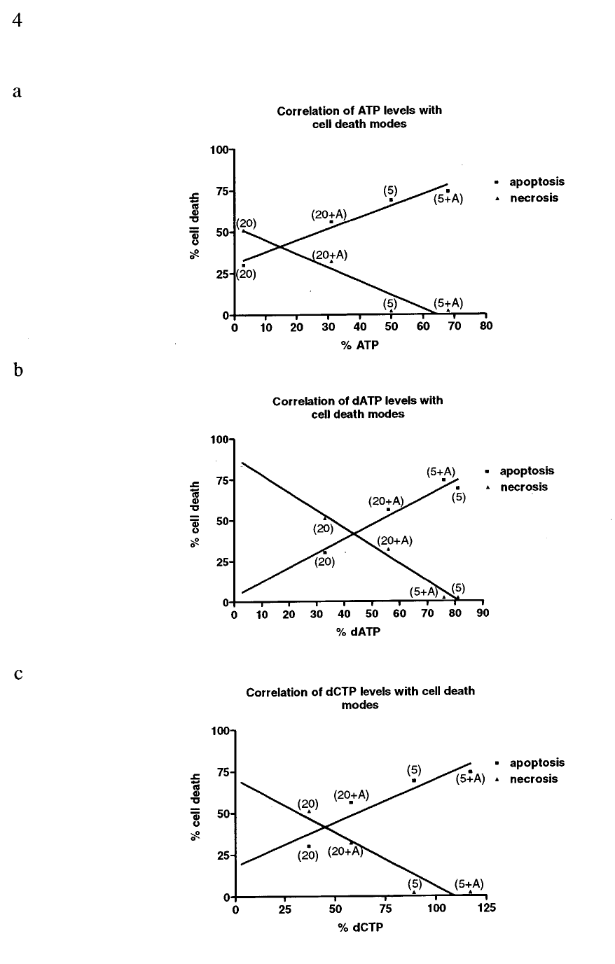

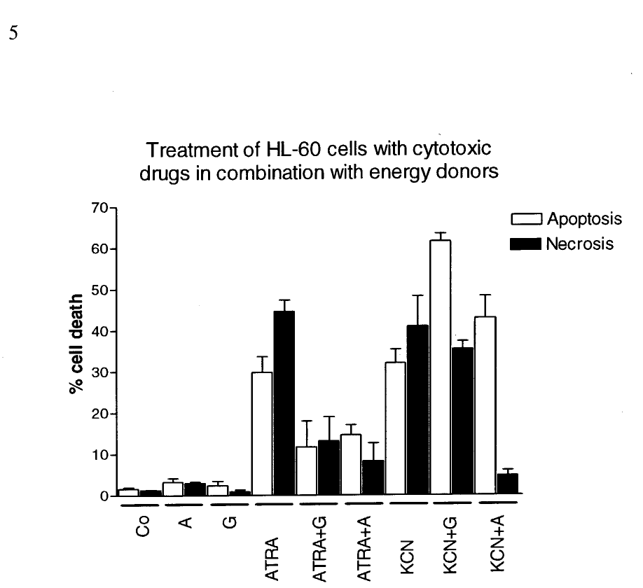

To examine whether prevention of necrosis by energy donors was a peculiarity of BR-induced cell death, or it represents a more general mechanism, HL-60 cells were treated with all-trans retinoic acid (ATRA) and potassium cyanide (KCN). ATRA is used clinically to treat acute promyeloic leukemia⁴⁰. KCN blocks the respiratory chain and prevents ATP generation⁴¹⁻⁴³. Exposure of the cells to 120 μM ATRA for 48 hours resulted in 30% apoptotic and 45% necrotic cells (apoptosis:necrosis ratio A:N=1:1.5) (FIG. 5). Addition of 100 mM glucose repressed both apoptosis and necrosis (A:N=1:1.2). Also, 800 μM adenosine repressed both types of cell death, but in this case the apoptosis rate was increased (A:N=1.8:1). The effect of glucose and adenosine on KCN-induced cell death was even more dramatic: 20 mM KCN alone caused 32% apoptosis, and 41% necrosis after 48 hours of treatment (A:N=1:1.3) in HL-60 cells. The addition of 100 mM glucose did not suppress KCN-induced cell death, as in the case with ATRA, and inverted the death ratio in favor of 61% apoptosis (A:N=1.7:1). 800 μM adenosine substantially suppressed necrosis and increased the apoptosis rate to 43% (A:N=9.1:1) (FIG. 5).

FIG. 5 shows the results of HL-60 cells which were treated with saline (Co), 100 mM glucose (G), 800 μM adenosine (A), 120 μM all-trans retinoic acid (ATRA), 20 mM potassium cyanide (KCN) and combinations of ATRA or KCN with glucose and adenosine for 48 hours. The type of cell death was determined by HOPI double staining. Statistical analysis by t-test confirmed that the difference between apoptosis-and necrosis-rates versus respective controls were significant (p<0.05).

Necrotic BR-Concentrations Induced DNA Double Strand Breaks

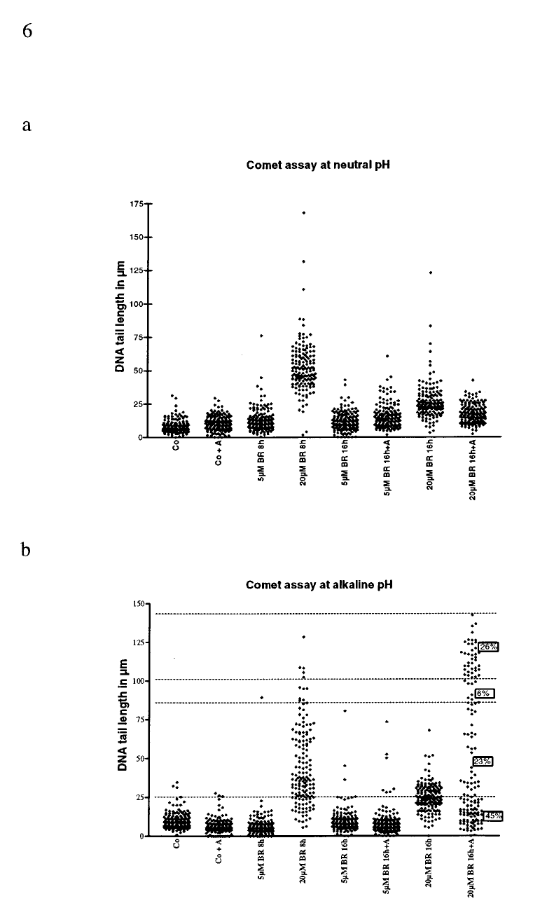

The DNA integrity was measured in individual cells with the single cell gel electrophoresis (comet) assay. The analyses were performed at time points (8 hours and 16 hours of treatment with BR), when the cell membranes were still intact and before apoptotic or necrotic markers were observed, but when ATP- and dNTP-pools were already effected. The results of comet assays performed at neutral pH (7.5) showed that 20 μM BR, but not 5 μM BR, induced DNA double strand breaks within 8 hours of treatment. These breaks were efficiently repaired after 16 hours (FIG. 6a). Additional comet-analyses at alkaline pH (13.0), demonstrated also that DNA single strand breaks occurred after incubation with 20 μM BR, but not with 5 μM BR treatment.

These lesions were substantially, but not completely repaired after 16 hours (FIG. 6b). It is conceivable, that the remaining single strand breaks might be the trigger for ATP depletion. (probably due to ongoing repair processes), and consequently for necrosis. These results also suggest that 5 μM BR-induced apoptosis is not causally related to DNA double or single strand breaks.

Viable and pre-apoptotic cells contain (relatively) high ATP levels in contrast to pre-necrotic cells. Therefore, cells with high-versus low ATP content were compared with the extent of DNA damage after 16 hours of treatment. Comet analysis at alkaline pH (13.0) revealed that the combined percentage of surviving+apoptotic cells (98% of the cells after each exposure to either 5 μM BR or 5 μM+adenosine), corresponded to a 25 μM DNA tail length (96% and 95%, respectively, see Table 2). Whereas, the percentage of necrotic cells (51% after treatment with 20 μM BR) correlated with cells with a >25 μm DNA tail length (50%) (Table 2, FIG. 6b). The inclusion of adenosine promoted DNA repair of a subset of 20 μM BR-treated cells after 16 hours. However, in 26% of the 20 μM BR-damaged cells, DNA-single strand breaks also accumulated after 16 hours when co-treated with adenosine, because DNA tail lengths increased (100 μm-140 μm) (FIG. 6b). The combined percentages (68%; viable+apoptotic) of 20 μM BR+adenosine treated cells did not correlate with the percentage of cells with a DNA tail length <25 μm (45%), (Table 2, FIG. 6b). This demonstrates that adenosine allowed ˜23% of the cells, which had substantially damaged DNA and, that were otherwise prone for necrosis (DNA tail length >25 μm, <87 μm), to undergo apoptosis.

FIG. 6 shows the induction of DNA damage in HL-60 cells by benzamide riboside (BR) cells were treated with saline (Co), adenosine (Co+A), 5 μM, and 20 μM BR, +/−adenosine, for 8 and 16 hours. The cells were then harvested for comet analysis at neutral (FIG. 6A) and alkaline (FIG. 6B) pH, and the extent of DNA migration was measured as described in “Methods”. Three cultures were made in parallel and from each culture 50 cells were evaluated. The figures show the distribution pattern of 150 cells. The values in rectangles (FIG. 6B) give the % of cells between the dotted lines, which were treated with 20 μM BR+adenosine. The observed differences in DNA tail length between Co and 20 μM BR treatment for 8 and 16 hours, +/−adenosine, are significant under neutral and alkaline conditions.

3-amino benzamide (3-AB) Represses necrosis

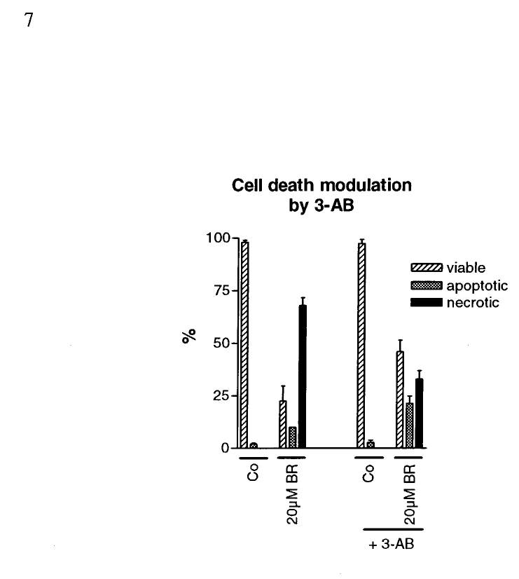

Since 20 μM BR dramatically depleted the ATP pool to 3% of control, it was speculated that this might have been due to DNA strand break-dependent activation of poly (ADP-ribose) polymerase (PARP). PARP consumes NAD and in consequence also affects the ATP pool^{44,45}. Moreover, PARP activity was shown to provoke necrosis^{34,46,47} and in PARP (−/−), mice upon cerebral ischemia reperfusion necrotic cell death did not occur⁴⁸. 3-AB is a potent inhibitor of PARP, and in fact, it was found that 20 μM BR-induced necrosis was inhibited in presence of 2 mM 3-AB (FIG. 7). This supports the assumption that PARP-activation might provoke BR-induced necrosis due to energy depletion, which is prevented by PARP-inhibition.

FIG. 7 shows the results of HL-60 cells, which were treated with saline (Co) and 20 μM BR, +/−3-amino benzamide (3-AB) for 48 hours. The cell status was analyzed by HOPI double staining. The reduced necrosis rates after treatment with 20 μM BR+3-AB are significant (p<0.05) whereas, the increased apoptosis rates are not (p=0.084).

Discussion

It is a well-known phenomenon that cytotoxic drugs, which can induce apoptosis and promote necrosis when administered at higher concentrations^{15-17,49}. Several reports suggest that apoptosis and necrosis share, in part, similar (early) pathways of induction^{17-19,50}. Also, p53 might determine whether a death pathway can be completed by apoptosis, or whether mutated p53 allows only for a necrotic fate at equitoxic concentrations.

It was suggested that the intracellular ATP level determines whether a cell dies in an apoptotic or necrotic mode^{15,34,36}. Therefore, ATP levels in benzamide riboside (BR)-treated HL-60 cells were investigated, which apoptosed after 5 μM treatments whereas, the cells underwent necrosis at 20 μM BR treatment. BR is a new synthetic C-nucleoside, which inhibits IMPDH, the rate-limiting enzyme of de novo guanylate biosyhthesis. IMPDH is frequently over-expressed in cancer cells and therefore, considered a target for anti-tumor therapy. It was previously demonstrated that BR exhibits strong antineoplastic activity in a panel of human tumor cell lines³ and was most effective in leukemia cells by inducing apoptosis^{13,14,51}. The oncolytic activity of BR³ was assumed to be due its IMPDH-inhibitory and, therefore, GTP and dGTP-limiting action^{2,12}. This hypothesis was strongly encouraged by the observation, that guanosine, a precursor of GTP and dGTP, prevented the oncolytic activity of BR^{12,52}.

The present findings show that 5 μM BR induced apoptosis, whereas, 20 μM BR provoked necrosis, although both concentrations of BR inhibited dGTP synthesis to a similar degree. dGTP levels were comparably affected by adenosine, which only marginally interfered with cell survival (see Table 2). Therefore, it seems unlikely that dGTP depletion alone accounted for the cell death mechanism elicited by BR. BR-mediated limitation of dGTP levels might result in less dGTP-mediated feed back stimulation of ADP reduction by ribonucleotide reductase (RR). This in consequence, decreases dADP levels and subsequently reduced dATP levels, which in fact was observed during treatment with BR. In turn, reduced dATP-mediated feedback inhibition of UDP-reduction by RR causes high dUDP and dUMP levels and this might be the reason for the observed increase in dTTP levels following BR treatment, because dUMP is the substrate for thymidylate synthase. Whereas, there was no dose response correlation between ATP- and dATP-levels to total cell death, there was a direct correlation of these nucleotide pools to apoptosis and an indirect correlation to necrosis. Thus, ATP and/or dATP levels seem to determine cell death modes as it was previously suggested by others^{15,35,36,50,53}.

In earlier investigations, it was demonstrated that BR suppressed survival pathways induced apoptosis-relevant genes^{13,14} and activated Caspase 8, but not Caspase 9 (Polgar et al., submitted). However, only low doses of BR (5 μM and 10 μM), but high doses (20 μM and more) induced apoptosis by a pathway that culminated in Caspase 3 activation. At high BR-concentrations the majority of cell death was by necrosis, presumably due to massive DNA damage. DNA double strand breaks became rapidly repaired, but a substantial amount of single strand breaks and/or alkali-labile sites remained non-repaired. Surprisingly, adenosine enabled a substantial number of the 20 μM BR-treated cells, which contained massive DNA damage (23%), to escape necrosis and to undergo an apoptotic pathway (Table 2; FIG. 6b). This percentage would correspond to cells with a DNA tail length between >25 μm and <87 μm.

DNA repair processes consume energy and also PARP, a repair enzyme that becomes activated in response to DNA strand breaks^{54,55} and, which uses NAD as a substrate⁵⁶. Finally, the cell depletes its ATP in an attempt to replenish its NAD pool^{34,44,45,55,57}. Thus, it is likely that the necrotic damage arising from high BR concentrations are a consequence of DNA strand breaks and subsequent loss of ATP, which would have been required for an orchestrated apoptotic program. Since maintenance of ATP by adenosine or supplementation with glucose, which is the major energy source of a cell, could prevent necrosis and favored apoptosis, therefore, ATP and possibly also dATP are determinants of cell death modes. This investigation supports the assumption that energy donors can promote apoptosis and repress necrosis when, cell death is induced by various cytotoxic and therapeutic agents.

### Example 2

Apoptosis eliminates unwanted cells without affecting the microenvironment, whereas, necrosis causes severe inflammation of surrounding tissues due to spillage of cell fluids into the pericellular space. In most cases, cytostatic treatment is limited by non-specific toxicity. Hence, anti-neoplastic drug administration has to be stringently controlled to avoid over-dosing, which otherwise causes necrotic-rather than apoptotic cell death. Therefore, benzamide riboside (BR), which exhibits strong oncolytic activity against leukemia cells in the 5-10 μM range, was tested. BR is a new bona fide inhibitor of inosine 5′-monophosphate dehydrogenase. In this experiment, the types of BR-induced cell deaths were quantified, which is of utmost importance for future in vivo studies. Higher concentrations (20 μM) predominantly induced necrosis, which correlated with DNA strand breaks and subsequent depletion of ATP and dATP levels due to the activation of DNA repair mechanisms. Artificial replenishment of the ATP pool by addition of adenosine prevented necrosis and favored apoptosis. This effect was not peculiarity of BR-treatment, but was reproduced with high concentrations of all-transretinoic acid (120 μM) and cyanide (20 mM). Glucose, the major cellular energy source, was also capable to suppress. necrosis and to favor apoptosis of HL-60 cells, which had been treated with necrotic doses of BR and cyanide. Thus, the monitoring and maintenance of cellular energy pools during therapeutic drug treatment should be taken into consideration, because this helps to minimize nonspecific side effects and to improve attempted drug effects.

Throughout this application, various publications, including United States patents, are referenced by author and year, and patents by number. Full citations for the publications are listed below. The disclosures of these publications and patents in their entireties are hereby incorporated by reference into this application, in order to more fully describe the state of the art to which this invention pertains.

The invention has been described in an illustrative manner, and it is to be understood that the terminology, which has been used, is intended to be in the nature of words of description, rather than of limitation.

Obviously, many modifications and variations of the present invention are possible in light of the above teachings. It is, therefore, to be understood that within the scope of the described invention, the invention may be practiced otherwise than as specifically described.

TABLE 2
Comparison of dNTP levels with DNA tail lengths and cell status

| | | | | | | | % viable + | | % total |
|---|---|---|---|---|---|---|---|---|---|
| | % ATP | % dATP | % dCTP | DNA tail length | % viable | % apopt | apopt | % necro | death |
| Control | 100 | 100 | 100 | 98% <= 25 μm | 98 | 2 | 100 | 0 | 2 |
| Ade | 85 | 70 | 64 | 98% <= 25 μm | 93 | 7 | 100 | 0 | 7 |
| 5 μM BR | 50 | 81 | 89 | 96% <= 25 μm | 29 | 69 | 98 | 2 | 71 |
| 20 μM BR | 3 | 33 | 37 | 50% <= 25 μm | 19 | 30 | 49 | 51 | 81 |
| | | | | 50% >= 25 μm | | | | | |
| 5 μM BR + Ade | 68 | 76 | 117 | 95% <= 25 μm | 24 | 74 | 98 | 2 | 76 |
| 20 μM BR + Ade | 31 | 56 | 58 | 45% <= 25 μm | 12 | 56 | 68 | 32 | 88 |
| | | | | 55% >= 25 μm | | | | | |

The percentages of ATP, dATP and dCTP levels and the DNA tail length of HL-60 cells after 16 hours of BR+/−adenosine treatment, and the percentages of viable, apoptotic and necrotic cells following treatment for 48 hours with BR+/−adenosine, were measured. Viable cells displayed nuclei homogenously stained with Hoechst and excluded PI. Apoptotic cells showed chromatin condensation and nuclear fragmentation. At least 200 cells were counted from each sample.

What is claimed is:

1. A composition comprising an ATP-inducing compound and an effective amount of a cell death-inducing drug.

2. The composition according to claim 1, wherein said cell death-inducing drug is an inosine 5′-monophosphate dehydrogenase inhibitor.

3. The composition according to claim 2, wherein said cell death-inducing drug is selected from the group consisting essentially of benzamide riboside and tiazofurin and any analogs thereof.

4. A composition comprising ATP or an ATP precursor and an effective amount of a cell death-inducing drug.

5. The composition according to claim 4, wherein said cell death-inducing drug is an inosine 5′-monophosphate dehydrogenase inhibitor.

6. The composition according to claim 5, wherein said cell death-inducing drug is selected from the group consisting essentially of benzamide riboside and tiazofurin.

7. A method of inducing apoptosis in apoptosis-inducible cells by administering to the apoptosis-inducible cells a composition comprising a cell death-inducing effective amount of a cell death-inducing compound and an ATP-inducing compound.

8. The method according to claim 7, wherein said administering step includes orally administering the composition.

9. The method according to claim 7, wherein said administering step includes intravenously administering the composition.

10. A method of inducing apoptosis in apoptosis-inducible cells by administering to the apoptosis-inducible cells a composition comprising a cell death-inducing effective amount of a cell death-inducing compound and ATP.

11. The method according to claim 10, wherein said administering step includes orally administering the composition.

12. The method according to claim 10, wherein said administering step includes intravenously administering the composition.

13. A method of suppressing necrosis in cells by administering an effective amount of an ATP-inducing compound to a cell population exhibiting necrosis.

14. The method according to claim 13, wherein said administering step includes orally administering the composition.

15. The method according to claim 13, wherein said administering step includes intravenously administering the composition.

16. A composition for inducing apoptosis in apoptosis-inducible cells, said composition comprising an ATP-inducing compound and an effective amount of a cell death-inducing drug.

17. The composition according to claim 16, wherein said cell death-inducing drug is an inosine 5′-monophosphate dehydrogenase inhibitor.

18. The composition according to claim 17, wherein said cell death-inducing drug is selected from the group consisting essentially of benzamide riboside and tiazofurin.

19. A composition for inducing apoptosis in apoptosis-inducible cells, said composition comprising ATP or an ATP precursor and an effective amount of a cell death-inducing drug.

20. The composition according to claim 19, wherein said cell death-inducing drug is an inosine 5′-monophosphate dehydrogenase inhibitor.

21. The composition according to claim 20, wherein said cell death-inducing drug is selected from the group consisting essentially of benzamide riboside and tiazofurin.
